(19) United States
(12) Patent Application Publication
(10) Pub. No.: US 2023/0048948 A1
(43) Pub. Date: Feb. 16, 2023

(54) TREATMENT INSTRUMENT

(71) Applicant: Olympus Medical Systems Corp., Tokyo (JP)

(72) Inventors: Tsubasa NIIYAMA, Tokyo (JP); Marina ASAHINA, Sagamihara-shi (JP); Hidenosuke HASE, Tokyo (JP)

(73) Assignee: Olympus Medical Systems Corp., Tokyo (JP)

(21) Appl. No.: 17/883,728

(22) Filed: Aug. 9, 2022

Related U.S. Application Data
(60) Provisional application No. 63/232,393, filed on Aug. 12, 2021.

(51) Int. Cl. A61B 18/14

(52) U.S. Cl. CPC A61B 18/14; A61B 2017/00389

(57) ABSTRACT

Treatment instrument includes an operation button for receiving a user operation provided in a housing. The housing has a pair of first surfaces spaced apart at predetermined intervals and a sliding portion of the operation button, provided with a curved second surface having a predetermined curvature, is located in the space between the pair of first surfaces and configured for rotation and to press a switch element in response to a user operation to affect an associated treatment operation.

### RELATED APPLICATION DATA

This application is based on and claims priority under 37 U.S.C. § 119 to U.S. Provisional Application No. 63/232,393 filed on Aug. 12, 2021, the entire contents of which are incorporated herein by reference.

### FIELD OF THE DISCLOSURE

The present invention relates to a treatment instrument.

### BACKGROUND

Conventionally, there has been known a treatment instrument which treats a site of interest by imparting treatment energy such as ultrasonic energy to a site to be treated in a biological tissue (hereinafter, referred to as a target site) (see, for example, Patent Document 1: JP 5,519,878).

The treatment instrument described in Patent Document 1 is provided with a housing and an operation button. The operation button is located on a side surface of the housing and, when an operator depresses the operation button, a treatment operation occurs. An example treatment operation imparts treatment energy to a target site.

In addition, in the treatment instrument described in Patent Document 1, the operation button can be manipulated in two different ways to control the treatment operation. In a first way of manipulation, the operation button moves in a direction along a linear first axis toward a switch element and the linear movement along the first axis causes a switch element to be pressed. In a second way of manipulation, the operation button moves in a direction intersecting the first axis and the operation button rotates about a second axis perpendicular to the first axis, and the rotational movement causes a switch element to be pressed.

### SUMMARY OF THE INVENTION

### Problem to be Solved by the Invention

However, in the treatment instrument described in Patent Document 1, in order to enable the second way of manipulation of the operation button, the operation button is pivotally supported with respect to the housing by a pin extending along the second axis. In other words, in the structure using a pin, since the number of members increases, the structure and operation of the instrument becomes complicated, and it is difficult to improve the ease of assembly of the treatment instrument.

In view of the above, it is an object of the present invention to provide a treatment instrument capable of simplifying structure and means of manipulation of the operation button and improved ease of assembly of the treatment instrument.

### Means for Solving the Problem

In order to solve the above-mentioned problems and achieve the purpose, a treatment instrument according to the present invention includes: a housing having a pair of first surfaces separated by a predetermined interval; an operation button disposed between the pair of first surfaces and having a sliding portion provided with a curved second surface having a predetermined curvature, the operation button configured to receive a user operation; and a switch element provided in the housing and configured to be pressed by the operation button in response to the user operation on the operation button, wherein the operation button is configured to be rotatable about a first axis while the second surface slides on the pair of first surfaces in a direction of a second axis, the first axis perpendicular to the second axis.

### Effect of the Invention

According to the treatment instrument according to the present invention, it is possible to simplify the structure and to improve the ease of assembly of the treatment instrument.

### BRIEF DESCRIPTION OF THE DRAWINGS

FIG. 1 is a diagram illustrating a treatment system according to an embodiment.

FIG. 2 is a diagram illustrating a configuration of an operation button.

FIG. 3 is a diagram illustrating a configuration of an operation button.

FIG. 4 is a diagram illustrating a configuration of an operation button.

FIG. 5 is a diagram showing the operation of the operation button.

FIGS. 6A and 6B are diagrams showing the operation of the operation button.

FIG. 7 is a diagram showing the operation of the operation button.

FIG. 8 is a diagram showing a modification of the configuration of the operation button.

### DETAILED DESCRIPTION

### Modes for Carrying Out the Invention

Hereinafter, embodiments for carrying out the present invention (hereinafter, embodiments) will be described with reference to the accompanying drawings. Note that the present invention is not limited by the embodiments described below. In addition, in the description of the drawings, the same parts are denoted by the same reference numerals.

Schematic Configuration of the Treatment System

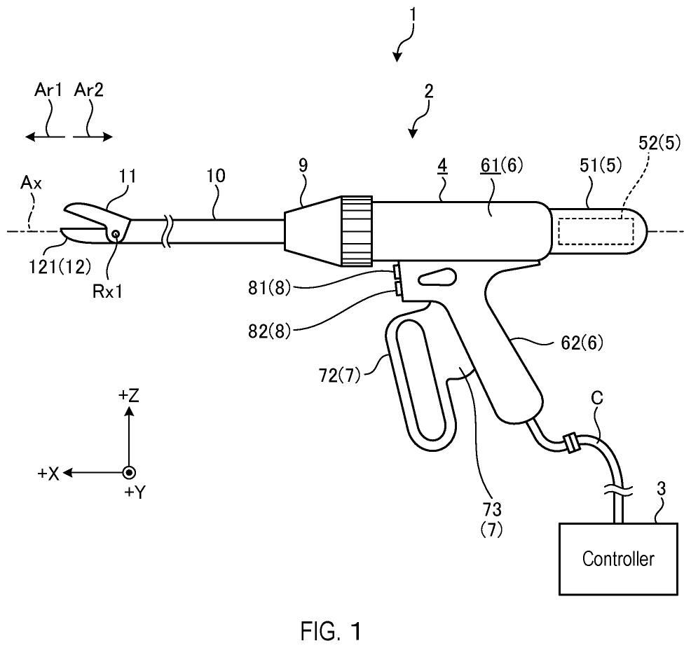

FIG. 1 is a diagram illustrating a treatment system 1 according to an embodiment. The treatment system 1 treats the target site by imparting treatment energy to a site to be treated in a biological tissue (hereinafter, described as a target site). Note that the treatment energy in the present embodiment is ultrasonic energy and high frequency energy, but embodiments can include other treatment energies, such as heat energies and microwave energies. Further, a treatment operation that can be performed by the treatment system 1 according to the present embodiment is a treatment operation such as coagulation (sealing) of a target site or incision of a target site, but other treatment operations can be conducted with the treatment system, such as anastomosis. In addition, treatment operations, such as coagulation and incision, may be performed simultaneously. The treatment system 1 comprises a treatment instrument 2 and a controller 3, as shown in FIG. 1.

Structure of the Treatment Instrument

In the following, in describing the configuration of the treatment instrument 2, the X-axis, Y-axis, and Z-axis are mutually orthogonal, as shown by the XYZ coordinate axis in FIG. 1. The X-axis is an axis parallel to the central axis Ax of the shaft 10 (FIG. 1), the Y-axis is an axis perpendicular to the plane of the paper, and the Z-axis is an axis along the vertical direction of FIG. 1. In addition, in the following, one side along the central axis Ax (+X-axis side) is described as a distal end side Ar1, and the other side (−X-axis side) is described as a proximal end side Ar2.

The treatment instrument 2 is an ultrasonic treatment instrument which treats the target site by imparting ultrasonic energy and high frequency energy to the target site. The treatment instrument 2 comprises a handpiece 4 and an ultrasonic transducer 5, as shown in FIG. 1.

The handpiece 4 includes a housing 6, a movable handle 7, first and second operation buttons 81 and 82, a rotation knob 9, a shaft 10, a jaw 11, and a vibration transmission member 12, as shown in FIG. 1. The housing 6 supports the entire treatment instrument 2. The housing 6, as shown in FIG. 1, has a substantially cylindrical case body 61, which is coaxial with the central axis Ax, extending from the case body 61 to the −Z-axis side (in FIG. 1, the lower side), and a fixed handle 62 which is gripped by an operator such as a medical professional. The movable handle 7 accepts a closing operation and an opening operation, which are user operations made by the operator. The movable handle 7, in response to the closing operation, moves in a direction close to the fixed handle 62. On the other hand, the movable handle 7, in response to the opening operation, moves in a direction away from the fixed handle 62.

First and second operation buttons 81 and 82, as shown in FIG. 1, are exposed to the outside from the side surface of the distal end-side Ar1 of the fixed handle 62 and are arranged aligned in the Z-axis direction. These first and second operation buttons 81,82 are operable by an operator to initiate a treatment operation. The treatment operation includes a procedure that imparts control energy to the site of interest.

In some embodiments, the first and second operation buttons 81 and 82 have substantially the same configuration. Then, if it is not necessary to distinguish the first and second operation button 81 and 82 in particular, then the first and second operation buttons 81 and 82 can together be described as operation button 8. The detailed configuration of the operation button 8 will be described later in the configuration of the operation button.

Rotary knob 9 has a substantially cylindrical shape or conical shape that is coaxial with the central axis Ax, as shown in FIG. 1, and is provided on the distal end-side Ar1 of the case body 61. The rotary knob 9 accepts a rotation control, which is a user operation by an operator. By the rotation control, the rotary knob 9 rotates about the central axis Ax with respect to the case body 61. Further, in addition to the rotary knob 9 being rotatable, the shaft 10, the jaw 11, and the vibration transmission member 12 also can be rotatable about the central axis Ax.

Shaft 10 is a cylindrical pipe made of a conductive material such as metal. Further, in the shaft 10, the end portion of the distal end-side Ar1 includes a jaw 11 and the jaw 11 is rotatably supported about a first rotational axis Rx1 extending in a direction perpendicular to the central axis Ax. Here, although not shown specifically in the housing 6 and the shaft 10, the jaw 11 has an opening and closing mechanism that causes elements of the jaw 11 to rotate around the first rotational axis Rx1 in response to the opening operation and closing operation affected by manipulation of the movable handle 7 by an operator. Then, by the opening and closing mechanism, the jaw 11 opens and closes with respect to the end portion 121 (hereinafter, referred to as the treatment portion 121 (FIG. 1)) on the distal end side Ar1 of the vibration transmission member 12 and grasps the target site between the jaw and the treatment portion 121. In addition, at least a part of the jaw 11 is made of a conductive material.

Vibration transmission member 12 is composed of a conductive material and has an elongated shape extending linearly along the central axis Ax. Further, the vibration transmission member 12, as shown in FIG. 1, in a state where the treatment portion 121 protrudes to the outside, is inserted into the shaft 10. The proximal end side Ar2 of the vibration transmission member 12 is mechanically connected to the ultrasonic vibrator 52 constituting the ultrasonic transducer 5. The vibration transmission member 12 transmits the ultrasonic vibration generated by the ultrasonic transducer 5 from the proximal end side Ar2 to the treatment portion 121. In the first embodiment, the ultrasonic vibration is a longitudinal vibration vibrating in a direction along the central axis Ax.

The ultrasonic transducer 5 includes a transducer (TD) case 51 and an ultrasonic transducer 52, as shown in FIG. 1. TD case 51 supports the ultrasonic vibrator 52 and is detachably connected to the case body 61. The ultrasonic vibrator 52 generates ultrasonic vibration under control by the controller 3. In the first embodiment, the ultrasonic vibrator 52 is constituted by a bolt-clamped Langevin transducer (BLT).

Composition of the Control Device

Controller 3 collectively controls the operation of the treatment instrument 2. Specifically, the controller 3, by passing operating signals through the electrical cable C, detects the operation of the operation button 8 by an operator. Then, when the controller 3 detects the button operation, operating signals and power are passed through the electric cable C to impart treatment energy to the target site grasped between the jaw 11 and the treatment portion 121. In other words, the controller 3 controls operation of the treatment instrument 2 to treat the target site.

For example, when applying ultrasonic energy to the target site, the controller 3 supplies drive power to the ultrasonic vibrator 52 by passing it through the electrical cable C. Thus, the ultrasonic vibrator 52 generates a longitudinal vibration (ultrasonic vibration) which vibrates in a direction along the central axis Ax. The treatment portion 121 also vibrates at a desired amplitude by the longitudinal vibration. Then, an ultrasonic vibration is applied from the treatment portion 121 to the target site grasped between the jaw 11 and the treatment portion 121. In other words, ultrasonic energy is applied from the treatment portion 121 to the target site.

Further, for example, when imparting high-frequency energy to the target site, the controller 3 supplies high-frequency power between the jaw 11 and the vibration transmission member 12 by passing it through the electric cable C. Thus, a high frequency current flows through the target site grasped between the jaw 11 and the treatment portion 121. In other words, the subject site is imparted with high frequency energy.

Additionally, the controller 3 can perform different control in the case of detecting the operation of the first operation button 81 and in the case of detecting the operation of the second operation button 82. Here, the different operation buttons can be used to impart only ultrasonic energy to the target site by operation of one of the operation buttons, and to impart only high frequency energy to the target site by operation of a second one of the operation buttons. Also, it is possible to impart both ultrasonic energy and high frequency energy to the target site by operation of both operation buttons.

Configuration of Operation Buttons

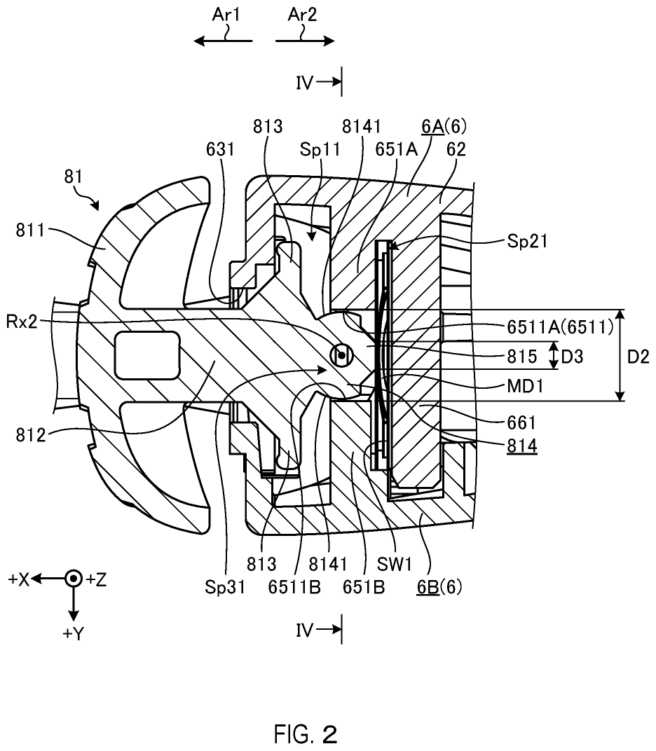

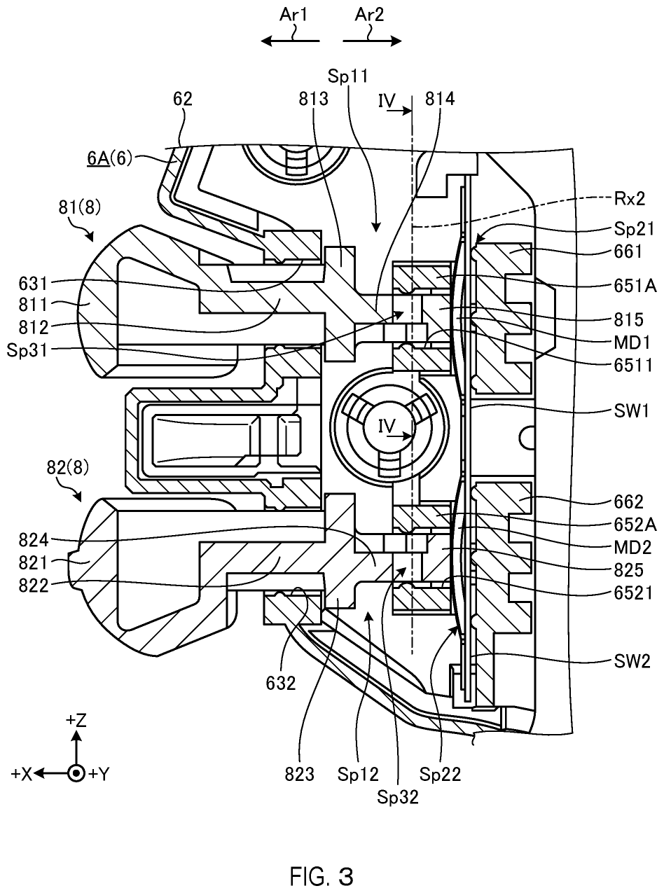

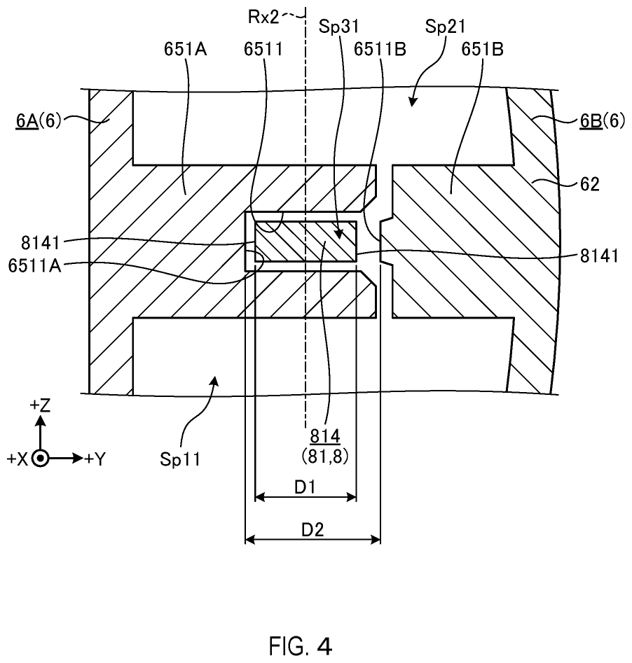

Next, a configuration of the operation button 8 is described. FIGS. 2 to 4 are views illustrating the configuration of the operation button 8. Specifically, FIG. 2 is a cross-sectional view of the first operation button 81 and the housing 6 in the XY plane passing through the first operation button 81. FIG. 3 is a cross-sectional view of the first operational button 81 and second operation button 82 in the housing 6 in the XZ plane passing through the first operational button 81 and second operation button 82. FIG. 4 is a cross-sectional view of the first operation button 81 and the housing 6 in the YZ plane taken at the position of IV-IV shown in FIGS. 2 and 3. Note that the position of IV-IV is a position passing through the second rotational axis Rx2.

First, a configuration of the housing 6 will be described before the configuration of the operation button 8 is described. The housing 6 is divided and formed into two bodies including a first one located on the −Y-axis side and a second one located on the +Y-axis side (with reference to the XZ plane). In other words, the housing 6 has a hollow shape by combining the portion on the −Y-axis side and the portion on the +Y-axis side. In the following, for convenience of explanation, the portion on the −Y-axis side is described as a first housing 6A (FIGS. 2 to 4), and the side on the +Y-axis side is described as a second housing 6B (FIGS. 2 and 4).

First, in a state where the first housing 6A and second housing 6B are combined, the housing 6 has an exterior surface and an internal cavity and, as shown in FIG. 2 and FIG. 3, a first communication hole 631 opens toward the distal end-side Ar1. The first operation button 81 passes through the first communication hole 631. Similarly, as shown in FIG. 3, a first communication hole 631 opens toward the distal end-side Ar1 and a second communication hole 632 opens toward the distal end-side Ar1. The first operation button 81 passes through the first communication hole 631 and The second operation button 82 passes through the first communication hole 632. the side surface of the distal end-side Ar1. The first communication hole 631 and second communication hole 632 are positioned at separate locations in the −Z-axis direction.

As shown in FIGS. 2 to 4, the inner surface of the first housing 6A includes a first protrusion 651A that protrudes linearly in the +Y-axis direction. As shown in FIGS. 2 to 4, the first protrusion 651A includes a first housing portion 6511 that is formed by a recess in the distal end surface of the first protrusion 651A. The first housing portion 6511 is recessed in a straight line in the −Y-axis direction and the recess is centered in the z-axis direction in the first housing portion 6511. The first housing portion 6511 can extend in the X-axis direction over the entire extent of the first protrusion 651A and forms a slot, as shown in FIG. 2 or FIG. 3. Then, the bottom surface of the recess forming the first housing portion 6511 corresponds to the first surface 6511A according to the present invention (FIGS. 2 and 4).

Further and as shown in FIG. 2 and FIG. 3, the inner surface of the first housing 6A also includes a first element mounting portion 661, which is located toward the proximal end side Ar2 relative to the first protrusion 651A. The first element mounting portion 661 protrudes linearly in the +Y-axis direction. The length dimension in the +Y-axis direction of the first element attachment portion 661 is larger than the length dimension in the +Y-axis direction of the first protrusion 651A.

Additionally and as shown in FIG. 3, the inner surface of the first housing 6A can also include a second protrusion 652A that is similar to the first protrusion 651A. The second protrusion 652A is positioned to the −Z-axis side relative to the first protrusion 651A. The second protrusion 652A has similar features to the first protrusion 651A. For example, features of the first protrusion 651A (including the first housing portion 6511 (including the first surface 6511A)) and the first element attachment portion 661 are similarly included in the second protrusion 652A, which includes the second housing portion 6521 (including the first surface (not shown)) and the second element attachment portion 662.

Also and as shown in FIG. 2 and FIG. 4, the inner surface of the second housing 6B includes a first protrusion 651B that protrudes linearly toward the −Y-axis direction. The first protrusion 651B of the second housing 6B is located at a position such that, when the first housing 6A is mated to the second housing 6B, the first protrusion 651B of the second housing 6B faces the first protrusion 651A of the first housing 6A. Also, when the first housing 6A is mated to the second housing 6B, the projecting end of the first protrusion 651B has a first surface 6511B that opposes the first surface 6511A of the first protrusion 651A. Further, the center (in the Z-axis direction) of the first surface 6511B is opposed to the center (in the Z-axis direction) of the first surface 6511A.

The inner surface of the second housing 6B has similar and/or complementary features to those of the inner surface of the first housing 6A, including a second protrusion (not shown). The second protrusion is disposed on the −Z-axis side of the first protrusion 651B of the second housing 6B. The second protrusion has similar features to those of the first protrusion 651B (including the first surface 6511B).

In the housing 6 described above, one or more (and preferably all) of the surfaces of the first housing 6A and of the second housing 6B that are in contact with the first sliding portion 814 and a second sliding portion 824 has a smaller surface roughness than other portions of the first housing 6A and second housing 6B not in contact with the first sliding portion 814 and the second sliding portion 824 in order that the first sliding portion 814 and the second sliding portion 824 move smoothly.

In the following and for convenience of explanation, in the fixed handle 62 portion of the housing 6, the portions of the space that are toward the distal end-side Ar1 relative to the pair of first protrusions 651A, 651B is described as a first distal end-side space Sp11 (FIG. 2, FIG. 3) and the portions of the space that are toward the distal end-side Ar1 relative to the pair of second protrusions 652A (second protrusion on second housing 6B not shown) is described as a second distal end-side space Sp12 (FIG. 3). Furthermore, in the fixed handle 62 portion of the housing 6, the space between the first element mounting portion 661 and the pair of first protrusions 651A, 651B is described as a first proximal space Sp21 (FIG. 2, FIG. 3). Further, in the fixed handle 62 portion of the housing 6, the space between the pair of second protrusions 652A (second protrusion on second housing 6B not shown) and the second element mounting portion 662 is described as a second proximal space Sp22 (FIG. 3). Further, a space formed by surfaces of the first housing portion 6511 and the first surface 6511B is described as a first housing space Sp31 (FIGS. 2 to 4). Further, a space formed by surfaces of the second housing portion 6521 and the surface of the protruding end of the second protrusion provided on the second housing 6B (not shown) is described as a second housing space Sp32 (FIG. 3).

As shown in FIG. 2 or FIG. 3, the first switch element SW1 is provided on a flexible substrate and is disposed in the first proximal space Sp21. This first switch element SW1 corresponds to a switch element according to the present invention and is a switch element having a metal dome MD1. The first switch element SW1 is disposed in the first proximal space Sp21 in a position in which the metal dome MD1 is directed toward the distal end side Ar1. When the first switch element SW1 is pressed by the first operation button 81 in response to operation of the first operation button 81 by an operator, an operation signal corresponding to the treatment operation associated with the first operation button 81 is outputted to the control device 3.

As shown in FIG. 3, the second switch element SW2 is provided on a flexible substrate and is disposed in the second proximal space Sp22. The second switch element SW2 corresponds to a switch element according to the present invention and is a switch element having a metal dome MD2. The second switch element SW2 is disposed in the second proximal space Sp22 in a position in which the metal dome MD2 is facing the distal end side Ar1. When the second switch element SW2 is pressed by the second operation button 82 in response to operation of the second operation button 82 by an operator, an operation signal corresponding to the treatment operation associated with the second operation button 82 is outputted to the control device 3.

FIGS. 2 to 4 show the first operation button 81, a first button body 811, a first connecting portion 812, a pair of first contact portion 813, a first sliding portion 814, and a first pusher portion 815. The first button body 811 is located outside the housing 6 and is a portion for receiving an operation by an operator. The first button body 811 has a shape in the X-Y plane extending in an arc around the second rotational axis Rx2.

As shown in FIG. 2 and FIG. 3, the first connecting portion 812 extends from the first button body 811 and connects the first button body 811 to the first sliding portion 814. The first connecting portion 812 passes through the first communication hole 631 and has a first portion located outside of the fixed handle 62 and a second portion located inside of the fixed handle 62.

A pair of first contact portions 813 corresponds to an abutment portion according to the present invention. As shown in FIG. 2, the pair of first contact portions 813 are located in the first distal end-side space Sp11. Within the pair of first contact portions 813, a first contact portion 813 protrudes from the first connecting portion 812 to the −Y-axis side and a second contact portion 813 protrudes from the first connecting portion 812 to the +Y-axis side, respectively. By abutting against the inner surface of the housing 6, the pair of first contact portions 813 has a function of preventing the first operation button 81 from escaping out of the housing 6.

The first sliding portion 814 corresponds to a sliding portion according to the present invention. As shown in FIG. 2 and FIG. 3, the first sliding portion 814 is provided at an end of the −X-axis side of the first connecting portion 812. The first sliding portion 814 has a flat plate shape extending along the XY plane. In the first sliding portion 814, the outer surface of the −Y-axis side and the outer surface of the +Y-axis side each has an arc shape around the second rotational axis Rx2. That is, the outer surface of the −Y-axis side and the outer surface of the +Y-axis side is formed into a curved surface having a predetermined curvature. The first sliding portion 814 is disposed in the first housing space Sp31, as shown in FIGS. 2 to 4. In the following, for convenience of description, the outer surface of the −Y-axis side and the outer surface of the +Y-axis side are described as a second surface 8141 (FIG. 2, FIG. 4). As shown in FIG. 4, the diameter dimension D1 of the second surface 8141 is smaller than the separation dimension D2 between the first surface 6511A of the first protrusion 651A and the first surface 6511B of the first protrusion 651B.

The first pusher portion 815 corresponds to the pusher portion according to the present invention. The first pusher portion 815 is provided at the end of the −X axis side of the first sliding portion 814. In some embodiments, the first pusher portion 815 is a protrusion that protrudes to the −X axis side of the first sliding portion 814. Also, the first pusher portion 815 is provided at a position so that, when assembled, the first pusher portion 815 is adjacent to the first switch element SW1 with respect to the first sliding portion 814. As shown in FIG. 2, the width dimension D3 of the projecting end of the first pusher portion 815 (length along the Y-axis direction) is smaller than the separation dimension D2 between the first surface 6511A of the first protrusion 651A and the first surface 6511B of the first protrusion 651B.

In the first operation button 81 described above, the outer surface of one or more of the first sliding portion 814 and the second sliding portion 824 that are in contact with the first housing 6A and the second housing 6B have a surface roughness that is smaller than other portions of the first operation button in order that the first sliding portion 814 and the second sliding portion 824 move smoothly.

As shown in FIG. 3, the second operation button 82 includes similar features to the first operation button 81, including a second button body 821, a second connecting portion 822, a pair of second contact portions 823, the second sliding portion 824 (including a second surface (not shown)), and a second pusher portion 825. The second connecting portion 822 extends from the second button body 821 and connects the second button body 821 to the second sliding portion 824. The second connecting portion 822 passes through the second communication hole 632 and has a first portion located outside of the fixed handle 62 and a second portion located inside of the fixed handle 62. Further, the pair of second contact portions 823 are respectively located in the second distal end-side space Sp12 and the second sliding portion 824 is disposed in the second housing space Sp32.

Action of Operation Button

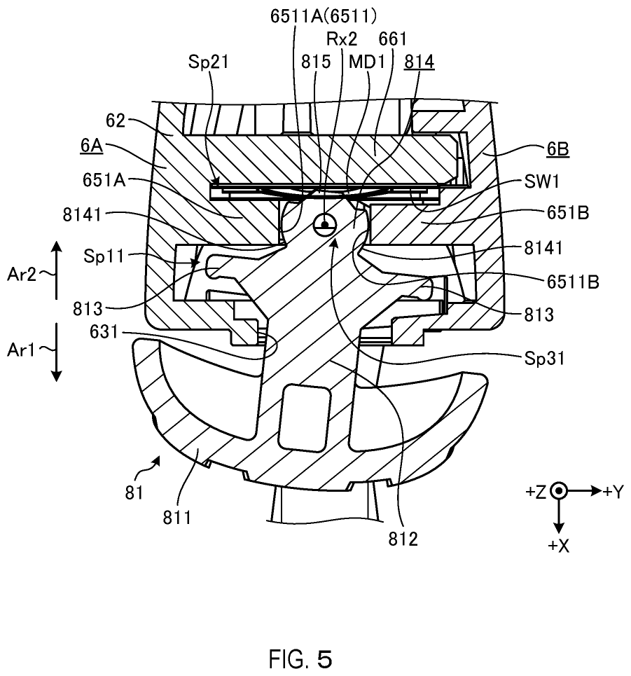

Next, operation of the operation button 8 corresponding to when an operator operates the operation button 8 to conduct a treatment operation will be described. FIGS. 5 to 7 are views illustrating the operation of the operation button 8. Specifically, FIGS. 5 to 7 are cross-sectional views corresponding to FIG. 2. Note that the operations of the first and second operation buttons 81,82 to conduct a treatment operation are substantially the same. For this reason, for convenience of description, an operation of the first operation button 81 will be described below.

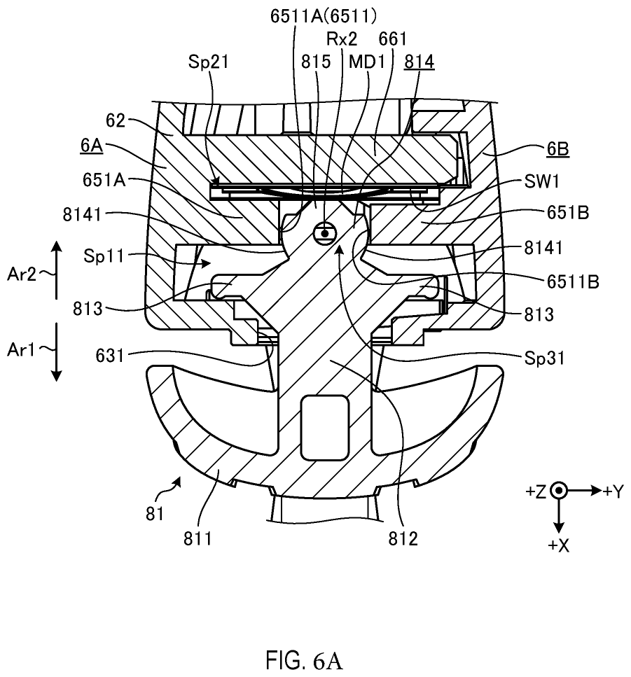

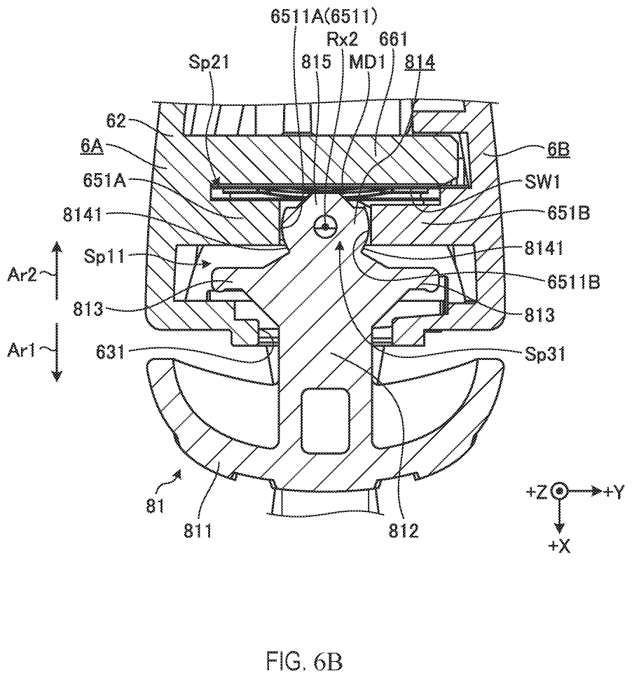

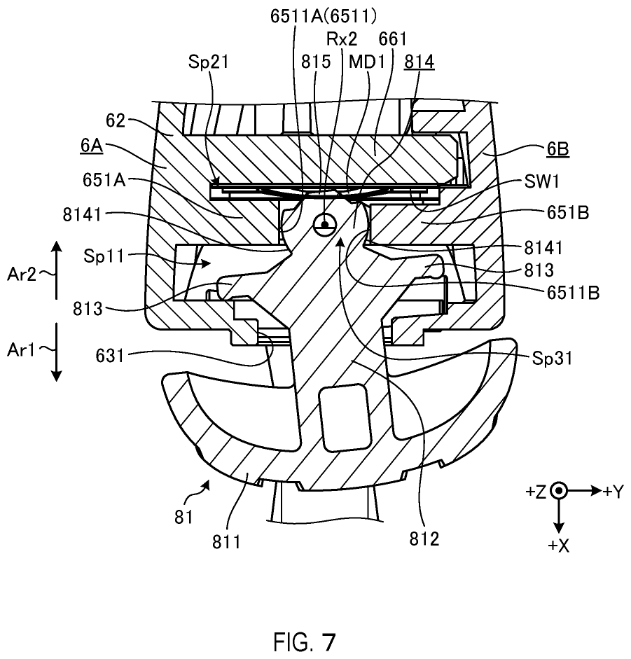

The operation button 8 can be operated by an operator in different ways to perform or of a first treatment operation and a second treatment operation (as described below). FIG. 6A shows a condition of the first operation button 81 in which the first and second treatment operations are not performed with respect to the first operation button 81, i.e., the first operation button 81 is in a neutral state. FIG. 6B shows the operation of the first operation button 81 according to the first treatment operation, i.e., the first operation button 81 is moved in a straight line along the X-axis toward the −X-axis side, and FIGS. 5 and 7 show the operation of the first operation button 81 according to the second treatment operation, i.e., the first operation button 81 is moved in the X-Y plane to a side of the X-axis.

The first treatment operation is an operation of pressing the first button body 811 in the first operation button 81 from the neutral state shown in FIG. 6A in a straight line along the X-axis toward the −X-axis side to the state shown in FIG. 6B. In the first treatment operation, the first operation button 81 moves linearly toward the −X-axis side, while the second surface 8141 slides on the first surface 6511A of the first protrusion 651A and on the first surface 6511B of the first protrusion 651B. The linear movement causes the first pusher portion 815 to depress the metal dome MD1 of the switch element. That is, the first switch element SW1 outputs an operation signal corresponding to the treatment operation associated with the first treatment operation. Here, the length dimension in the X-axis direction of the first surfaces 6511A, 6511B is larger than the amount of movement of the first operation button 81 in the first treatment operation.

After movement of the first operation button 81 to cause the first treatment operation described above, when an operator releases his/her hand from the first operation button 81, the first operation button 81 is biased toward the +X-axis side by the reaction force of the metal dome MD1, and the first operation button 81 returns to the neutral state shown in FIG. 6A. In the neutral state shown in FIG. 6A, the first operation button 81 is in contact with the inner surface of the distal end-side Ar1 of the housing 6 and movement of the first operation button 81 to the +X-axis side is restricted. For example, the pair of first contact portions 813 can be in contact with the inner surface at substantially parallel positions in the YZ plane.

The second treatment control is an operation of pressing the first button body 811 of the first operation button 81 toward the −Y-axis side or +Y-axis side, relative to the neutral state shown in FIG. 6A. FIG. 5 shows the state of the first operation button 81 positioned for the second treatment operation by pressing toward the −Y-axis side and FIG. 7 shows the state of the first operation button 81 positioned for the second treatment operation by pressing toward the +Y-axis side.

As shown in FIG. 5 or FIG. 7, in the second treatment operation, the first operation button 81 has moved such that one of the pair of first contact portions 813 of the first operation button 81 is in contact with the inner surface of the distal end-side Ar1 in the housing 6, preferably in the position in the YZ plane as in the neutral state, and the other of the pair of first contact portions 813 of the first operation button 81 is not in contact with the inner surface of the distal end-side Ar1 in the housing 6. Also, in the second treatment operation, the second surface 8141 slides on the first surface 6511A of the first protrusion 651A and the first surface 6511B of the first protrusion 651B as the first operation button 81 rotates about the second rotational axis Rx2. The movement of the first operation button 81 in the second treatment operation causes the first pusher portion 815 to depress the metal dome MD1 of the switch element. That is, the first switch element SW1 outputs an operation signal corresponding to the treatment operation associated with the second treatment operation.

Here, at least a portion of the first sliding portion 814 is disposed between the pair of first surface 6511A, 6511B in both a state in which the treatment control is not performed with respect to the first operation button 81 (FIG. 6A), and a state in which the treatment operation is performed with respect to the first operation button 81 (FIGS. 5, 6B, and 7). Further, the second rotational axis Rx2 is the rotational center of the first operation button 81 and is located between the first surface 6511A of the first protrusion 651A and the first surface 6511B of the first protrusion 651B.

After movement of the first operation button 81 to cause the second treatment operation described above, when an operator releases his/her hand from the first operation button 81, the first operation button 81 is biased toward the +X-axis side by the reaction force of the metal dome MD1, and returns to the neutral state shown in FIG. 6A.

According to the present embodiment described above, the following effects can be achieved.

In the treatment tool 2 according to the present embodiment, the first operation button 81 is configured to be rotatable by the user operation about the second rotation axis Rx2 by sliding the second surface 8141 in the first sliding portion 814 located between the pair of first surfaces 6511A, 6511B on the pair of first surfaces 6511A, 6511B. In other words, in this embodiment, the configuration of the second surface 8141 in the first sliding portion 814 located between the pair of first surfaces 6511A, 6511B constrains the first operation button 81 so that it rotates relative to the neutral state upon pressing the first button body 811 of the first operation button 81 toward the −Y-axis side or +Y-axis side. Further, the rotation of the first operation button 81 in the second treatment operation is fixed without use of a pin at the second rotation axis Rx2, as is used in the conventional configuration. Therefore, in the treatment instrument 2 according to the present embodiment, since a conventional pin is not used, it is possible to reduce the number of parts, to simplify the structure, and to improve the ease of assembly.

In addition, in the treatment tool 2 according to the present embodiment, in the housing 6, one or more (and preferably all) of the surfaces of the first housing 6A and of the second housing 6B that are in contact with the first sliding portion 814 and the second sliding portion 824 has a smaller surface roughness than other portions of the first housing 6A and second housing 6B not in contact with the first sliding portion 814 and the second sliding portion 824, and the outer surface of one or more of the first sliding portion 814 and the second sliding portion 824 that are in contact with the first housing 6A and the second housing 6B have a surface roughness that is smaller than other portions of the first operation button. Therefore, when the first operation button 81 is operated to cause the first and second treatment operations, the sliding resistance between the first operation button 81 and the housing 6 is relatively low, and it is possible to improve the operability of the first and second treatment operations.

### Other Embodiments

While embodiments for carrying out the present invention have been described so far, the present invention is not to be limited only by the embodiments described above.

In the above-described embodiment, as the treatment instrument according to the present invention, a configuration is set to impart both ultrasonic energy and high frequency energy to the target site, but the present invention is not limited thereto. As the treatment instrument according to the present invention, it may be employed a configuration that imparts at least one treatment energy of ultrasonic energy, high frequency energy, and thermal energy to a target site. By “imparting heat energy to a target site” is meant that heat generated in a heater or the like is transmitted to a target site.

In the above-described embodiment, the operation button 8 is used as an output button, but the present invention is not limited thereto, and may be configured by a button having another function such as a button for performing an operation of discharging a fluid such as a liquid or a gas toward a living tissue.

In the embodiment described above, the two operation buttons 8 are provided, a first operation button 81 and a second operation button 82. But the present invention is not limited thereto, and only one operation button 8 may be provided, or alternatively, three or more operation buttons 8 may be provided.

In the embodiment described above, a switch element having a metal dome has been adopted, for example, the first switch element SW1 has a metal dome MD1 and a second switch element SW2 has a metal dome MD2. But the switch element(s) are not limited thereto and other switch elements having no metal dome may be adopted.

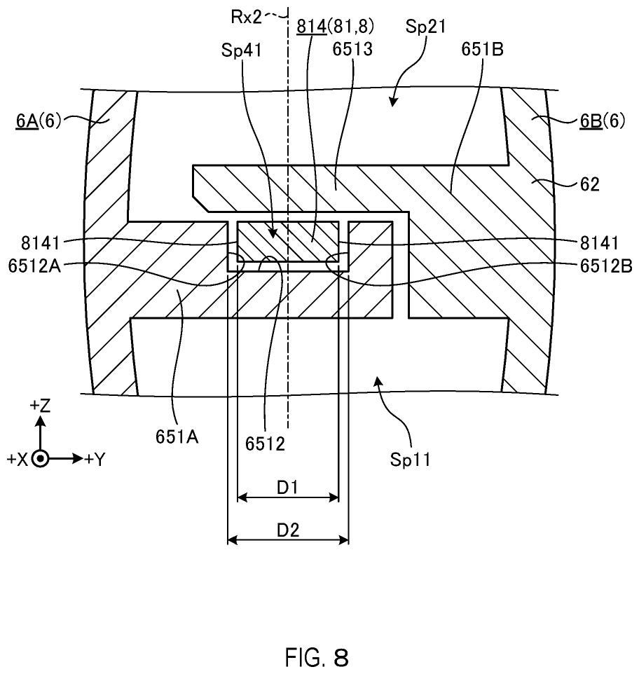

FIG. 8 is a diagram showing a modification of the above-described embodiment. Specifically, FIG. 8 is a cross-sectional view corresponding to FIG. 4 and shows another embodiment. In the above-described embodiment, the first surface 6511A is provided on the first protrusion 651A and the first surface 6511B is provided on the first protrusion 651B, but the present invention is not limited thereto. Rather, in another embodiment, the configuration of the pair of first surfaces 6511A, 6511B may be provided only on one of the pair of first protrusions 651A, 651B. In the modification shown in FIG. 8, a pair of first surfaces 6511A, 6511B is provided only in the first protrusion 651A of the pair of first protrusions 651A, 651B.

Specifically, in the first protrusion 651A according to the present modification, the first housing portion 6511 described in the above-described embodiment is omitted. Further and as shown in FIG. 8, in the first protrusion 651A, the +Z-axis side surface in the first protrusion 651A, the first housing portion 6512 is recessed toward the −Z-axis side. The first housing portion 6512 penetrates the first protrusion 651A in the X-axis direction. Then, a pair of side surfaces intersecting the Y-axis in the first housing portion 6512 corresponds to the first surfaces 6512A, 6512B according to the present invention (compare to FIG. 4).

Further and as shown in FIG. 8, in this modification the first protrusion 651B protrudes toward the −Y-axis side and a portion 6513 of the first protrusion 651B extends past the opening in the first housing portion 6512 and covers the +Z-axis side of the opening. Then, the first sliding portion 814 is disposed in the first housing space Sp41 (FIG. 8) surrounded by the first storage portion 6512 and the portion 6513 of the first protrusion 651B.

### DESCRIPTION OF SYMBOLS

1 Treatment system
2 treatment instrument
3 Controller
4 Handpiece
5 Ultrasonic transducer
6 Housing
6A first housing
6B second housing
7 Movable handle
8 Operation button
9 Rotary knob
10 Shaft
11 Jaw
12 Vibration transmission member
51 Transducer case
52 Ultrasonic vibrator
61 Case body
62 Fixed handle
81 First operation button
82 Second operation button
121 Treatment portion
631 First communication hole
632 Second communication hole
651A, 651B first protrusion
652A second protrusion
661 First element mounting portion
662 Second element mounting portion
811 First button body
812 First connecting portion
813 First contact portion
814 First sliding portion
815 First pusher portion
821 Second button body
822 Second connecting portion
823 Second contact portion
824 Second sliding portion
825 Second pusher portion
6511, 6512 First housing portion
First aspect of 6511A, 6511B, 6512A, 6512B
6513 Portions
6521 Secondary housing portion
8141 Second surface
Ar1 distal end side
Ar2 proximal end side
Ax central axis
C Electrical cable
D1 Diameter dimension of the first sliding portion
D2 Spacing dimension between the first protrusions
D3 Width dimension of the tip of the first pusher portion
MD1,MD2 metal dome
Rx1 first rotational axis
Rx2 second rotational axis
Sp11 first distal end-side space
Sp12 second distal end-side space
Sp21 first proximal space
Sp22 second proximal space
Sp31, Sp41 first housing space
Sp32 second housing space
Sw1 first switch element
Sw2 second switch element

What is claimed is:

1. A treatment instrument, comprising:
a housing having an outer surface and an inner surface, wherein the inner surface includes a pair of first protrusions and wherein one of the first protrusions include a recess having plurality of surfaces defining a recess, wherein two surfaces of the plurality of surfaces defining the recess oppose each other and are separated by a predetermined distance;
an operation button configured to receive a user operation, wherein the operation button includes a sliding portion and wherein the sliding portion has an outer surface having a curvature an arc shape around a rotational axis Rx2; and
a switch provided in the housing, wherein the switch is configured to be pressed by the operation button in response to the user operation received by the operation button;
wherein, in response to the user operation, the operation button is configured to rotate about the rotational axis when the outer surface of the sliding portion of the operation button slides on the two surfaces of the plurality of surfaces defining the recess.

2. The treatment instrument according to claim 1, wherein the operation button further includes a pusher portion and wherein the pusher portion is configured to press the switch element.

3. The treatment instrument of claim 2, wherein the operation button has a first end and a second end,
wherein the first end is connected to the second end by a connecting portion, and
wherein the first end includes a first button body and the second end includes the sliding portion and the pusher portion.

4. The treatment instrument of claim 2, wherein a width (D3) of the pusher portion is less than the predetermined distance separating the opposing two surfaces of the plurality of surfaces defining the recess.

5. The treatment instrument according to claim 3, wherein the pusher portion is a protrusion that protrudes from the second end of the first button body and has a base part and a tip part,
wherein the base part is oriented toward the connecting portion and is wider than the tip part, and
wherein a width of the tip part is less than the predetermined distance separating the opposing two surfaces of the plurality of surfaces defining the recess.

6. The treatment instrument according to claim 1, wherein the curvature of the outer surface of the sliding portion has an arc shape and is located at a constant radial distance from a rotational axis Rx2, and
wherein a length of the radial distance is less than the predetermined distance separating the opposing two surfaces of the plurality of surfaces defining the recess.

7. The treatment instrument according to claim 3, wherein the operation button further includes a plurality of contact portions which extend outward from the connecting portion, and
wherein the plurality of contact portions each contact the inner surface of the housing.

8. The treatment instrument according to claim 7, wherein the operation button has a first surface roughness, an outer surfaces of the plurality of contact portions of the operation button have a second surface roughness, and
wherein remaining surfaces of the operation button have a surface roughness that is greater than the first surface roughness and is greater than the second surface roughness.

9. The treatment instrument according to claim 1, wherein the opposing two surfaces of the plurality of surfaces defining the recess have a third surface roughness, and
wherein remaining portions of the inner surface of the housing have a surface roughness that is greater than the third surface roughness.

10. The treatment instrument according to claim 1, wherein the operation button is configured to move linearly toward the switch by the outer surface of the sliding portion of the operation button sliding on the two surfaces of the plurality of surfaces defining the recess, and
wherein a length of the outer surface of the sliding portion of the operation button in a direction corresponding to the linear movement of the operation button is greater than a distance by which the operation button linearly moves.

11. The treatment instrument according to claim 1, wherein at least a portion of the outer surface of the sliding portion of the operation button are located between the opposing two surfaces of the plurality of surfaces defining the recess when the operation button is in a neutral state and when the operation button is in first state associated with a first treatment operation and in a second state associated with a second treatment operation.

12. The treatment instrument according to claim 1, wherein a rotation center (Rx2) of the operation button is located between the two surfaces of the plurality of surfaces defining the recess.

13. The treatment instrument according to claim 1, wherein the operation button is exposed to an outer surface of the housing at a distal end side (Ar1) of the housing.

14. The treatment instrument according to claim 1, wherein the treatment device includes a plurality of operation buttons.

15. The treatment instrument according to claim 1, wherein the treatment instrument is configured to treat a biological tissue by imparting a treatment energy to the biological tissue,
wherein the treatment energy is at least one of an ultrasonic energy and a radiofrequency energy, and
wherein the operation button is an output button for initiating application of the treatment energy to the biological tissue.
